(19) United States
(12) Patent Application Publication
(10) Pub. No.: US 2023/0109972 A1
(43) Pub. Date: Apr. 13, 2023

(54) SOLVENT COMPOSITIONS WITH ANTIOXIDANTS

(71) Applicant: Dow Global Technologies LLC, Midland, MI (US)

(72) Inventors: Felipe A. Donate, Midland, MI (US); Rebecca J. Wachowicz, Bay City, MI (US)

(21) Appl. No.: 17/760,370

(22) PCT Filed: Mar. 1, 2021
(86) PCT No.: PCT/US2021/020226

§ 371 Date: Aug. 9, 2022

Related U.S. Application Data
(60) Provisional application No. 63/014,773, filed on Apr. 24, 2020.

(51) Int. Cl. C09K 15/08; C11B 5/00; C07C 43/04

(52) U.S. Cl. CPC C09K 15/08; C11B 5/0035; C07C 43/04

(57) ABSTRACT

The present invention relates to solvent compositions and processes for inhibiting the generation of peroxide in glycol ether solvents. In one aspect, a solvent composition comprises at least one glycol ether, at least one antioxidant containing a hindered phenol, wherein the concentration of the antioxidant in the solvent composition is less than or equal to 1,000 ppm by weight, and a peroxide, wherein the concentration of the peroxide in the solvent composition is less than 20 ppm, and wherein the APHA color index of the solvent composition is less than 25.

### FIELD

The present invention relates to antioxidants for use in treating solvents and in particular, to solvent compositions having a glycol ether solvent and at least one antioxidant. The present invention also relates to processes for inhibiting the generation of peroxide in glycol ether solvents.

### BACKGROUND

Glycol ethers and glycol diethers (collectively referred to herein as “glycol ethers”) are among a class of solvents known to form peroxides on exposure to air. Glycol ether peroxides tend to decompose into aldehydes and carboxylic acids, which eventually results in the glycol ether solvents not meeting desired assay, acidity and color specifications. The formation of peroxides in glycol ethers can be curtailed by the addition of inhibitors, such as butylatedhydroxytoluene (BHT), which is known to be effective as an antioxidant at low parts per million levels. For this reason, BHT is widely used today with many known glycol ether solvents to provide the needed peroxide inhibition to the glycol ether solvents.

DOWANOL™ DPnB (dipropylene glycol n-butyl ether) is one of the known glycol ether solvents that is inhibited with BHT. DOWANOL™ DPnB containing BHT is a solvent that is used in a variety of cleaning products. And, DOWANOL™ DPnB with BHT is listed in the CleanGredients database of chemical ingredients that have been pre-approved by the US EPA to meet the Safer Choice Standard criteria. However, a recent decision by the EPA to remove compounds inhibited with BHT from the above list will cause the delisting of DOWANOL™ DPnB with BHT as well as other solvents that use BHT as an inhibitor, unless a BHT alternative can be implemented in the solvent. It would be desirable to have a BHT alternative antioxidant for such solvents.

### SUMMARY

It has been surprisingly found that several antioxidants such as d,l-alpha-tocopherol, d,l-beta-tocopherol, d,l-gamma-tocopherol, d,l-delta-tocopherol, and n-propyl gallate, at low concentrations (e.g., from 50 ppm to 1000 ppm by weight), can prevent peroxide and acid generation in glycol ethers. Therefore, the present invention is directed to the use of low concentrations of one or more of the above antioxidants in glycol ether solvents to provide a peroxide inhibited glycol ether solvent composition.

In one aspect, a solvent composition of the present invention comprises: (a) at least one glycol ether having the following formula:

R₁—O—(CHR₂CHR₃)O)ₙR₄;

wherein R₁ is an alkyl group having 1 to 9 carbon atoms or a phenyl group; wherein R₂ and R₃ each individually is hydrogen, a methyl group or an ethyl group, provided that when R₃ is a methyl group or an ethyl group, R₂ is hydrogen and provided that when R₂ is a methyl group or an ethyl group, R₃ is hydrogen; wherein R₄ is hydrogen or an alkyl group having 1 to 4 carbon atoms; and wherein n is an integer of 1 to 3;

(b) at least one antioxidant containing a hindered phenol, wherein the amount of the antioxidant in the solvent composition is less than or equal to 1,000 ppm by weight based on the total weight of the solvent composition; and

(c) a peroxide, wherein the amount of the peroxide in the solvent composition is less than 20 ppm, based on the total weight of the solvent composition,

wherein the APHA color index of the solvent composition is less than 25.

In another aspect, the present invention relates to a process for inhibiting the generation of peroxide in glycol ether solvents, the process comprising treating at least one glycol ether solvent with at least one antioxidant to reduce the content of a peroxide in the solvent to provide a glycol ether solvent composition, wherein the at least one glycol ether has the following formula:

R₁—O—(CHR₂CHR₃)O)ₙR₄;

wherein R₁ is an alkyl group having 1 to 9 carbon atoms or a phenyl group; wherein R₂ and R₃ each individually is hydrogen, a methyl group or an ethyl group, provided that when R₃ is a methyl group or an ethyl group, R₂ is hydrogen and provided that when R₂ is a methyl group or an ethyl group, R₃ is hydrogen; wherein R₄ is hydrogen or an alkyl group having 1 to 4 carbon atoms; and wherein n is an integer of 1 to 3; wherein the at least one antioxidant contains a hindered phenol; wherein the concentration of the antioxidant in the solvent composition is less than or equal to 1,000 ppm by weight, based on the total weight of the solvent composition; wherein the concentration of the peroxide in the solvent composition is less than 20 ppm, based on the total weight of the solvent composition; and wherein the APHA color index of the solvent composition is less than 25.

In another embodiment, the present invention relates to a glycol ether solvent composition prepared using any of the inventive processes disclosed herein.

These and other embodiments are described in more detail in the Detailed Description.

### DETAILED DESCRIPTION

As used throughout this specification, the abbreviations given below have the following meanings, unless the context clearly indicates otherwise: “<” means “less than”; “>” means “greater than”; “≤” means “less than or equal to”; ≥” means “greater than or equal to”; “@” means “at”; μm=micron(s), g=gram(s); mg=milligram(s); L=liter(s); mL=milliliter(s); g/mL=gram(s) per milliliter; ppm=parts per million; ppmw=parts per million by weight; rpm=revolutions per minute; m=meter(s); mm=millimeter(s); cm=centimeter(s); min=minute(s); s=second(s); hr=hour(s); ° C.=degree(s) Celsius; mmHg=millimeters of mercury; psig=pounds per square inch; kPa=kilopascal(s); %=percent, vol %=volume percent; and wt %=weight percent.

Some embodiments of the present invention relate to solvent compositions. In one embodiment, a solvent composition comprises: (a) at least one glycol ether having the following formula:

R₁—O—(CHR₂CHR₃)O)ₙR₄;

wherein R₁ is an alkyl group having 1 to 9 carbon atoms or a phenyl group; wherein R₂ and R₃ each individually is hydrogen, a methyl group or an ethyl group, provided that when R₃ is a methyl group or an ethyl group, R₂ is hydrogen and provided that when R₂ is a methyl group or an ethyl group, R₃ is hydrogen; wherein R₄ is hydrogen or an alkyl group having 1 to 4 carbon atoms; and wherein n is an integer of 1 to 3;

(b) at least one antioxidant containing a hindered phenol, wherein the amount of the antioxidant in the solvent composition is less than or equal to 1,000 ppm by weight based on the total weight of the solvent composition; and

(c) a peroxide, wherein the amount of the peroxide in the solvent composition is less than 20 ppm, based on the total weight of the solvent composition,

wherein the APHA color index of the solvent composition is less than 25.

In some embodiments, the glycol ether is selected from the group consisting of dipropylene glycol methyl ether; propylene glycol n-butyl ether; dipropylene glycol n-butyl ether; tripropylene glycol n-butyl ether; propylene glycol n-propyl ether; dipropylene glycol n-propyl ether; propylene glycol methyl ether acetate; dipropylene glycol methyl ether acetate; dipropylene glycol dimethyl ether; and mixtures thereof. In some embodiments, the amount of glycol ether in the solvent composition is 95 weight percent to 99.99 weight percent, based on the total weight of the solvent composition.

In some embodiments, the at least one antioxidant is selected from the group consisting of selected from the group consisting of d,l-alpha-tocopherol, d,l-beta-tocopherol, d,l-gamma-tocopherol, d,l-delta-tocopherol, n-propyl gallate, and mixtures thereof. The at least one antioxidant, in some embodiments, is d,l-alpha-tocopherol. In some embodiments, the amount of antioxidant in the solvent composition is 50 ppm to 1,000 ppm, based on the total weight of the solvent composition.

In some embodiments, the concentration of peroxide present in the glycol ether solvent composition is less than 10 ppm, based on the total weight of the solvent composition.

The glycol ethers used in the present invention are usually highly pure compounds but can include mixtures of the homologous series. It is well known that when an alcohol is reacted with ethylene oxide or propylene oxide to produce a glycol ether, a mixture of products containing one, two or three ethylene oxide or propylene oxide units is obtained. This mixture is separated by distillation into the main components but sometimes a small quantity of one homolog can remain in a given product.

In some embodiments, the glycol ether(s) useful in the present invention is used at a purity of from 95% to 99.99% and can include one or more of the aforementioned homologue compounds. Typically, some commercial embodiments of glycol ether are mixtures of the homologues.

Examples of some commercially available glycol ethers useful in the present invention include, for example, products commercially available under the tradename DOWANOL™ from The Dow Chemical Company, such as propylene glycol n-butyl ether (DOWANOL™ PnB), dipropylene glycol n-butyl ether (DOWANOL™ DPnB), tripropylene glycol n-butyl ether (DOWANOL™ TPnB), propylene glycol n-propyl ether (DOWANOL™ PnP), dipropylene glycol n-propyl ether (DOWANOL™ DPnP), propylene glycol methyl ether acetate (DOWANOL™ PMA), dipropylene glycol methyl ether acetate (DOWANOL™ DPMA), and dipropylene glycol dimethyl ether (PROGLYDE™ DMM); and mixtures thereof.

The concentration of the glycol ether, component (a), in solvent compositions of the present invention can be, for example, 90 weight percent to 99.99 weight percent in some embodiments, or from 95 wt % to 99.99 wt % in some embodiments, based on the total weight of the solvent composition.

The antioxidant, component (b), used in embodiments of the present invention can include one or more compounds, including, for example, d,l-alpha-tocopherol, d,l-beta-tocopherol, d,l-gamma-tocopherol, d,l-delta-tocopherol, n-propyl gallate (propyl, 3,4,5-trihydroxybenzoate) and mixtures thereof.

In one embodiment, the antioxidant comprises d,l-alpha tocopherol. In one embodiment, the antioxidant comprises d,l-alpha-tocopherol, d,l-beta-tocopherol, d,l-gamma-tocopherol, d,l-delta-tocopherol, and mixtures thereof.

Non-limiting examples of commercially available antioxidants that can be used in embodiments of the present invention include Irganox E201, which is d,l-alpha tocopherol commercially available from BASF, and Covi-Ox T90, which is d,l-alpha tocopherol (and also includes d,l-beta-tocopherol, d,l-gamma-tocopherol, and d,l-delta-tocopherol) commercially available from BASF.

By including such antioxidants in the solvent compositions of the present invention, the one or more antioxidants can reduce both the concentration of peroxides present in the glycol ether and the acidity of the glycol ether.

The amount of the one or more antioxidants, component (b), that can be used in solvent compositions of the present invention can be, for example, 50 ppm to 1,000 ppm by weight in one embodiment, 50 ppm to 250 ppm by weight in other embodiments, and 90 ppm to 110 ppm by weight in other embodiments, based on the total weight of the solvent composition. If the concentration of the antioxidant is above 1,000 ppm, the antioxidant can generate color in the glycol ether; and if the concentration of the antioxidant is below 50 ppm in some embodiments, the antioxidant can fail to provide sufficient peroxide inhibition in the glycol ether.

The present invention also relates to processes for inhibiting the generation of peroxide in glycol ether solvents. In one embodiment, a process for inhibiting the generation of peroxide in glycol ether solvents comprises treating at least one glycol ether solvent with at least one antioxidant to reduce the content of a peroxide in the solvent to provide a glycol ether solvent composition, wherein the at least one glycol ether has the following formula:

R₁—O—(CHR₂CHR₃)O)ₙR₄;

wherein R₁ is an alkyl group having 1 to 9 carbon atoms or a phenyl group; wherein R₂ and R₃ each individually is hydrogen, a methyl group or an ethyl group, provided that when R₃ is a methyl group or an ethyl group, R₂ is hydrogen and provided that when R₂ is a methyl group or an ethyl group, R₃ is hydrogen; wherein R₄ is hydrogen or an alkyl group having 1 to 4 carbon atoms; and wherein n is an integer of 1 to 3; wherein the at least one antioxidant contains a hindered phenol; wherein the concentration of the antioxidant in the solvent composition is less than or equal to 1,000 ppm by weight, based on the total weight of the solvent composition; wherein the concentration of the peroxide in the solvent composition is less than 20 ppm, based on the total weight of the solvent composition; and wherein the APHA color index of the solvent composition is less than 25. The at least one glycol ether can include any of the glycol ethers (component (a)) discussed above. The antioxidant, in some embodiments, can include one or more of d,l-alpha-tocopherol, d,l-beta-tocopherol, d,l-gamma-tocopherol, d,l-delta-tocopherol, n-propyl gallate (propyl, 3,4,5-trihydroxybenzoate), and mixtures thereof as discussed above.

The treating of the glycol ether with the antioxidant can be carried out in a number of ways including, for example, combining, blending or mixing (a) the at least one glycol ether and (b) the at least one antioxidant. The mixing of the components can be carried by any well-known means and process equipment known in the art of compounding products together. For example, an aliquot of the desired weight of antioxidant can be added from a shot tank or pumped into a reactor or storage tank containing the glycol ether and allowed to mix well. The addition of the antioxidant to the glycol ether can be carried out at ambient temperature (about 25° C.) and ambient pressure (about 760 mmHg or 14.7 psig).

The amount of time used in the process to treat the glycol ether solvent with the antioxidant and fully inhibit the solvent can depend on a number of factors including the antioxidant used, the glycol ether to be treated, the technique used for treating, mixing speed, and other factors. The glycol ether is treated with antioxidant for at least 10 minutes in some embodiments, at least 25 minutes in some embodiments, at least 30 minutes in some embodiments, up to 60 minutes in some embodiments, from 10 to 50 minutes in some embodiments, from 10 to 30 minutes in some embodiments. In general, lower treatment times should be avoided so as to avoid improper dissolution of the antioxidant, but longer times would not cause issues.

Some advantageous benefits/improvements of using processes of the present invention include, for example, some antioxidants contemplated herein (e.g., the tocopherols) are liquids and can be easily added and mixed with the glycol ether. In contrast, the prior antioxidant, BHT, is a solid and requires considerable mixing to dissolve BHT in glycol ether.

The resulting inhibited glycol ether solvent composition after undergoing the inhibition treatment as described herein has a residual concentration of undesirable peroxide present in the glycol ether of <25 ppm by weight in some embodiments, from 0 ppm to 15 ppm by weight in other embodiments, and from 0.01 ppm to 10 ppm by weight in still another embodiment, each based on the total weight of the solvent composition. The residual concentration of peroxide present in the glycol ether is measured using the technique described in the Examples section herein. Glycol ethers have a peroxide specification since peroxides can interfere undesirably with some of the solvent applications.

In addition, the resulting glycol ether solvent composition after undergoing the treatment described above can have several beneficial properties including for example, control of peroxides, reduced acidity, and/or having an APHA color index of <25.

The inhibited glycol ether solvent compositions prepared as described herein can be advantageously useful in various applications including, for example, solvent-based and water-based formulations for cleaners, paints, personal care products, among many other products.

### Examples

The following examples are presented to further illustrate the present invention in detail but are not to be construed as limiting the scope of the claims. Unless otherwise indicated, all parts and percentages are by weight.

Various terms and designations used in the Inventive Examples (Inv. Ex.) and the Comparative Examples (Comp. Ex.) are explained as follows:

The various antioxidants compounds and solvents used in the Inv. Ex. and the Comp. Ex. are described as follows:

### Antioxidants and Solvents

A number of potential antioxidants are identified as being potentially compatible with glycol ether solvents and are selected for evaluation in a one-week accelerated stability test at 60° C. in the presence of air. The following nine antioxidants are selected for the evaluation: (1) propyl gallate, (2) citric acid, (3) sorbic acid, (4) 1,2-octanediol, (5) gluconolactone, (6) ascorbic acid, (7) 4-hydroxyacetophenone, (8) d,l-α-tocopherol (available from BASF as Irganox E201), and (9) Covi-Ox T90 (a natural vitamin E which includes d,l-alpha-tocopherol, d,l-beta-tocopherol, d,l-gamma-tocopherol, d,l-delta-tocopherol and is available from BASF).

A preliminary solubility determination is first conducted to determine if the antioxidants are soluble in glycol ether solvents. Those antioxidants found to be compatible with the glycol ether solvents are further evaluated in the 0 ppm to 1,000 ppm concentration range.

The above nine antioxidants are evaluated with the following seven glycol ether solvents, available under the tradename DOWANOL™ from The Dow Chemical Company: (1) propylene glycol n-butyl ether (DOWANOL™ PnB, “PnB”), (2) dipropylene glycol n-butyl ether (DOWANOL™ DPnB, “DPnB”), (3) tripropylene glycol n-butyl ether (DOWANOL™ TPnB, “TPnB”), (4) propylene glycol n-propyl ether (PnP), (5) dipropylene glycol n-propyl ether (DOWANOL™ DPnP, “DPnP”), (6) propylene glycol methyl ether acetate (PMA), and (7) dipropylene glycol methyl ether acetate (DOWANOL™ DPMA, “DPMA”). The antioxidants are also evaluated in dipropylene glycol dimethyl ether (PROGLYDE™ DMM, “DMM”), a glycol diether solvent commercially available from The Dow Chemical Company. To the extent that the commercial versions of these solvents included BHT, the BHT is removed from the solvents by distillation such that the solvents evaluated in these Examples are not inhibited with BHT.

The antioxidants are evaluated for the antioxidants' ability to prevent or minimize peroxide formation and acid generation in the solvents. The performance of a solvent containing an antioxidant is compared against the performance of: (1) an uninhibited product and (2) a product inhibited with 100 ppm of BHT.

General Procedure for Removing Peroxides from Solvents

Samples of uninhibited (i.e., no BHT) DOWANOL™ DPnP, DOWANOL™ DPnB, and DOWANOL™ TPnB are procured. These samples contained >50 ppm peroxides. Therefore, the peroxides had to be removed from the solvents before these solvents could be inhibited with the test inhibitors. The peroxides are effectively removed from the above listed uninhibited solvent samples by passing the solvents through a chromatographic column packed with: (i) about 20 mL of sand, (ii) 20 mL of Celite diatomaceous earth, and (iii) 300 mL of activated alumina. The above packed chromatographic column (packed in the order of (i)-(iii)) was capable of removing peroxides from the solvents to a concentration of <1 ppm from a solvent quantity of 2 liters. After each of the peroxide-free uninhibited solvents is collected from the peroxide removal process, each of the solvents is promptly inhibited with a desired concentration of a test inhibitor.

### General Procedure for Testing Inhibitors

Inhibitors are tested as follows: a desired amount of inhibitor is weighed to four decimal places into a 250-mL round bottom flask equipped with a built-in thermocouple well. A 70 mL aliquot of the test solvent is measured in a graduated cylinder and added to the tared flask. The weight of solvent is recorded. A Teflon stir bar is added, and the flask is attached to a reflux condenser open to the air and secured to the hood lattice. A heating mantle connected to a digital temperature controller is attached to the flask, and a stirring plate is placed beneath the mantle. Control and high temperature limit thermocouples leading from the temperature controller are placed between the flask and the mantle. A thermocouple leading from a separate digital temperature meter is placed inside the thermocouple well. The stirrer is started, and the temperature controller set to control the mantle temperature at about 80° C. to 90° C., which is the mantle temperature required to maintain the solvent in the flask at 60° C.±1° C. This procedure is repeated with other identical experimental set ups that ran in parallel. After one week had elapsed, the heating is discontinued and the solvents are allowed to cool to room temperature before measuring the solvents' peroxide and acid content.

### Analytical Procedures

### Percent Acidity Measurements

Sample acidity is measured as per the method described in ASTM D1613. The ASTM D1613 method requires a sample to be mixed with isopropanol and then titrated to the phenolphthalein end point of the sample with dilute sodium hydroxide. The general procedure used to measure the acid content of a solvent is as follows:

A 50-mL aliquot of anhydrous isopropanol is added into an oven-dried 250-mL Erlenmeyer flask containing a Teflon stir bar. Six drops of 0.5% alcoholic phenolphthalein are added to the flask using a disposable pipette. Then, the alcohol in the flask is titrated to a faint pink end point with 0.02 N NaOH solution. A 25-mL aliquot of a test sample of solvent is pipetted into the aforementioned flask, and the above-described titration repeated. The volume of NaOH solution used for the sample titration is recorded and used to calculate the acidity, as weight percent (wt %) acetic acid (AA), according to the following equation:

Wt % AA=(mL of NaOH solution)×(Normality of NaOH solution)×0.12/sample density (g/mL).

The solvent sample densities at 25° C. used in the above equation are as follows: PnB=0.875 g/mL; DPnB=0.907 g/mL; TPnB=0.927 g/mL; PnP=0.880 g/mL; DMM=0.899 g/mL; DPnP=0.916 g/mL; PMA=0.963 g/mL; and DPMA=0.974 g/mL.

### Peroxide Content Measurements

The peroxide content of test solvent samples is measured with an analytical method based on reaction of the peroxide with iodide ion followed by titration of liberated iodine with thiosulfate. This procedure is used to measure the peroxide content in glycol ether solvent compositions as described herein and as recited in the claims. The procedure used to measure the peroxide content of a solvent is as follows:

A 35.0 g sample of the test solvent is weighed into a 250-mL Erlenmeyer flask, followed by the addition of 100 mL of anhydrous isopropanol and 10 mL of glacial acetic acid. A blank is prepared by adding 100 mL of anhydrous isopropanol and 10 mL of glacial acetic acid into another 250-mL Erlenmeyer flask. A 1-mL aliquot of a saturated potassium iodide (KI) solution that had been previously prepared by mixing 201.9 g KI crystals with 168.3 g of deionized water, is pipetted into each flask, and a stopper is placed on each flask. The flasks are shaken lightly and then placed in a dark cabinet for 5 min. The flasks are then removed from the dark cabinet and a Teflon stir bar is added into the flasks. The contents of each flask are then titrated sequentially with 0.005 N sodium thiosulfate (prepared by dilution of a commercial 0.1 N sodium thiosulfate) until the solvent's yellow color disappeared as determined by visual observation. An observed solvent having a light yellow color is usually indicative of the solvent having a relatively low peroxide concentration which can be titrated with a low volume of the dilute sodium thiosulfate solution. An observed solvent having a dark yellow color is very likely indicative of the solvent having a high peroxide concentration. In this case, a non-diluted 0.1 N sodium thiosulfate is used as the titrant instead of the 0.005 N sodium thiosulfate. The peroxide concentration of the solvent is calculated as ppm peroxide according to the following equation:

Peroxide (ppm)=[(mL NaS₂O₃ solution for sample being tested−mL NaS₂O₃ solution for a blank sample)×N NaS₂O₃×8,000]/g sample

Initial Evaluation of Antioxidants with PnB

Initial experiments are conducted with PnB samples containing different inhibitors. Peroxide concentrations measured in PnB after one week are described in Table I. Gluconolactone and ascorbic acid are insoluble in PnB even at 50 ppm. Therefore, Gluconolactone and ascorbic acid are removed from the tests. A peroxide concentration of 1,131 ppm is measured in the uninhibited solvent, whilst only 1.4 ppm peroxide is found in the BHT-inhibited solvent. The solvent inhibited with 1,2-octanediol or 4-hydroxyacetophenone contained high levels of peroxides, 713 ppm and 1,193 ppm respectively, which indicated little or no antioxidant performance in the solvent. Citric acid and sorbic acid limited peroxide formation to 43 ppm and 154 ppm, respectively; however, the citric acid and sorbic acid are not as efficient as d,l-alpha-tocopherol or n-propyl gallate which controlled peroxide formation at <1 ppm. The above results showed that not all compounds work well in glycol ether solvents as antioxidants. In view of the above initial evaluation results, d,l-alpha-tocopherol and n-propyl gallate are further evaluated at concentrations as low as 50 ppm (described in Table I); and d,l-alpha-tocopherol and n-propyl gallate are found to effectively control peroxide formation even at the low concentration of 50 ppm.

TABLE I
Peroxide Concentration (ppm) Measured
in PnB after One week at 60° C.

| | Inhibitor Concentration (ppm) | | | | | |
|---|---|---|---|---|---|---|
| Inhibitor | 0 | 50 | 100 | 250 | 500 | 1,000 |
| BHT | 1,131 | — | 14 | — | — | — |
| d,l-α-tocopherol | — | 0.9 | 1.1 | 1.5 | 1.7 | 0.5 |
| propyl gallate | — | 3.0 | 3.7 | 3.1 | 3.0 | 0.6 |
| citric acid | — | — | — | — | — | 43 |
| sorbic acid | — | — | — | — | — | 154 |
| 1,2-octanediol | — | — | — | — | — | 713 |
| gluconolactone | — | — | insoluble | — | — | — |
| asorbic acid | — | — | insoluble | — | — | — |
| 4-hydroxyacetophenone | — | — | — | — | — | 1,193 |

Acid concentrations measured in the various PnB samples are described in Table II. High acid concentrations are found in samples that had high peroxide concentrations. The acid concentrations in the solvent samples inhibited with d,l-alpha-tocopherol are as low as the levels found in solvent inhibited with BHT (0.0003 wt %). Given the superior performance of d,l-alpha-tocopherol, this compound is selected for further testing as an antioxidant in other glycol ether solvents.

TABLE II
Acidity (Weight % Acetic Acid) Measured in PnB after One Week at 60° C.

| | Inhibitor Concentration (ppm) | | | | | |
|---|---|---|---|---|---|---|
| Inhibitor | 0 | 50 | 100 | 250 | 500 | 1,000 |
| BHT | 0.0343 | — | 0.0003 | — | — | — |
| d,l-α-tocopherol | — | 0.0003 | 0.0003 | 0.0003 | 0.0003 | 0.0012 |
| propyl gallate | — | 0.0005 | 0.0005 | 0.0033 | 0.0063 | 0.0137 |
| citric acid | — | — | — | — | — | 0.0291 |
| sorbic acid | — | — | — | — | — | 0.0217 |
| 1,2-octanediol | — | — | — | — | — | |
| gluconolactone | — | — | insoluble | — | — | — |
| asorbic acid | — | — | insoluble | — | — | — |
| 4-hydroxyacetophenone | — | — | — | — | — | 0.0535 |

Evaluation of Other Glycol Ethers with Antioxidants

Seven glycol ethers and one glycol diether (DMM), which had been previously distilled and/or treated with activated alumina to remove BHT and peroxides, are inhibited with d,l-alpha-tocopherol and evaluated in the same one-week test at 60° C. as described above. The solvents PnB, DPnB, DPnP, PnP, and PMA are evaluated with antioxidant concentrations ranging from 0 to 250 ppm (see Table III). These tests showed that an antioxidant concentration of 100 ppm effectively controlled the formation of peroxides. Additional tests conducted with solvents TPnB, DPMA, and DMM showed that 100 ppm of d,l-alpha-tocopherol is also an effective antioxidant concentration for these solvents. The tests are repeated with all eight solvents inhibited with 100 ppm of the antioxidant Covi-Ox T90 (see Table III). These tests using Covi-Ox T90 as the antioxidant showed that this antioxidant is also capable of controlling peroxide formation. Acid values measured in the glycol ether samples inhibited with d,l-alpha-tocopherol or Covi-Ox T90 are orders of magnitude lower than the acid values resulting from the uninhibited samples (see Table IV). The results described in Table IV show effective acidity control by both the d,l-alpha-tocopherol antioxidant and the Covi-Ox T90 antioxidant.

TABLE III
Peroxide Concentrations (ppm) measured in Various Glycol
Ethers after One Week at 60° C.

| Glycol | | Inhibitor Concentration (ppm) | | | |
|---|---|---|---|---|---|
| Ether | Inhibitor | 0 | 50 | 100 | 250 |
| PnB | d,l-α-tocopherol | 1,131 | 0.09 | 1.1 | 1.5 |
| | Covi-Ox T90 | | — | 91 | — |
| DPnB | d,l-α-tocopherol | 470 | 4 | 4 | 3 |
| | Covi-Ox T90 | | — | 71 | — |
| DPnP | d,l-α-tocopherol | 548 | 3.6 | 3 | 5 |
| | Covi-Ox T90 | | — | 18 | — |
| PnP | d,l-α-tocopherol | 1,051 | 15.7 | 6.6 | 1.7 |
| | Covi-Ox T90 | | — | 110 | — |
| PMA | d,l-α-tocopherol | 98 | — | 3.3 | 3.3 |
| TPnB | d,l-α-tocopherol | 240 | — | 6.1 | — |
| | Covi-Ox T90 | | — | 84.5 | — |
| DPMA | d,l-α-tocopherol | 139 | — | 1.4 | — |
| | Covi-Ox T90 | | — | 1.3 | — |
| DMM | d,l-α-tocopherol | 315 | — | 1.7 | — |
| | Covi-Ox T90 | | — | 1.8 | — |

TABLE IV
Acidity as Weight % Acetic Acid Measured in Glycol
Ethers after 60° C. Test

| Glycol | | Inhibitor Concentration (ppm) | | | |
|---|---|---|---|---|---|
| Ether | Inhibitor | 0 | 50 | 100 | 250 |
| PnB | d,l-α-tocopherol | 0.0343 | 0.0003 | 0.0003 | 0.0003 |
| | Covi-Ox ® T90 | | — | 0.0005 | — |
| DPnB | d,l-α-tocopherol | 0.9950 | 0.0005 | 0.0011 | 0.0005 |
| | Covi-Ox ® T90 | | — | 0.0008 | — |
| DPnP | d,l-α-tocopherol | 0.4760 | 0.0005 | 0.0005 | 0.0005 |
| | Covi-Ox ® T90 | | — | 0.0008 | — |
| PnP | d,l-α-tocopherol | 0.1784 | 0.0005 | 0.0005 | 0.0005 |
| | Covi-Ox ® T90 | | — | 0.0005 | — |
| PMA | d,l-α-tocopherol | 0.0155 | — | 0.0060 | 0.0062 |
| TPnB | d,l-α-tocopherol | 0.6858 | — | 0.0526 | — |
| | Covi-Ox ® T90 | | — | 0.0401 | — |
| DPMA | d,l-α-tocopherol | 0.0862 | — | 0.0010 | — |
| | Covi-Ox ® T90 | | — | 0.0012 | — |
| DMM | d,l-α-tocopherol | 0.4346 | — | 0.0008 | — |
| | Covi-Ox ® T90 | | — | 0.0005 | — |

1. A solvent composition comprising:
(a) at least one glycol ether having the following formula:
R₁—O—(CHR₂CHR₃)O)ₙR₄;
wherein R₁ is an alkyl group having 1 to 9 carbon atoms or a phenyl group; wherein R₂ and R₃ each individually is hydrogen, a methyl group or an ethyl group, provided that when R₃ is a methyl group or an ethyl group, R₂ is hydrogen and provided that when R₂ is a methyl group or an ethyl group, R₃ is hydrogen; wherein R₄ is hydrogen or an alkyl group having 1 to 4 carbon atoms; and wherein n is an integer of 1 to 3;
(b) at least one antioxidant containing a hindered phenol, wherein the amount of the antioxidant in the solvent composition is less than or equal to 1,000 ppm by weight based on the total weight of the solvent composition; and
(c) a peroxide, wherein the amount of the peroxide in the solvent composition is less than 20 ppm, based on the total weight of the solvent composition,
wherein the APHA color index of the solvent composition is less than 25 and wherein the amount of glycol ether present in the solvent composition is 95 weight percent to 99.99 weight percent, based on the total weight of the solvent composition.

2. The solvent composition of claim 1, wherein the at least one glycol ether is selected from the group consisting of dipropylene glycol methyl ether; propylene glycol n-butyl ether; dipropylene glycol n-butyl ether; tripropylene glycol n-butyl ether; propylene glycol n-propyl ether; dipropylene glycol n-propyl ether; propylene glycol methyl ether acetate; dipropylene glycol methyl ether acetate; dipropylene glycol dimethyl ether; and mixtures thereof.

3. The solvent composition of claim 1, wherein the at least one antioxidant is selected from the group consisting of d,l-alpha-tocopherol, d,l-beta-tocopherol, d,l-gamma-tocopherol, d,l-delta-tocopherol, n-propyl gallate, and mixtures thereof.

4. The solvent composition of claim 1, wherein the at least one antioxidant is d,l-alpha-tocopherol.

5. The solvent composition of claim 1, wherein the amount of peroxide present in the solvent composition is less than 10 ppm by weight, based on the total weight of the solvent composition.

6. The solvent composition of claim 1, wherein the antioxidant inhibitor concentration in the solvent composition is 50 ppm to 1,000 ppm by weight, based on the total weight of the solvent composition.

7. (canceled)

8. A process for inhibiting the generation of peroxide in glycol ether solvents comprising treating at least one glycol ether solvent with at least one antioxidant to reduce the content of a peroxide in the solvent to provide a glycol ether solvent composition, wherein the at least one glycol ether has the following formula:
R₁—O—(CHR₂CHR₃)O)ₙR₄;
wherein R₁ is an alkyl group having 1 to 9 carbon atoms or a phenyl group; wherein R₂ and R₃ each individually is hydrogen, a methyl group or an ethyl group, provided that when R₃ is a methyl group or an ethyl group, R₂ is hydrogen and provided that when R₂ is a methyl group or an ethyl group, R₃ is hydrogen; wherein R₄ is hydrogen or an alkyl group having 1 to 4 carbon atoms; and wherein n is an integer of 1 to 3; wherein the at least one antioxidant contains a hindered phenol; wherein the concentration of the antioxidant in the solvent composition is less than or equal to 1,000 ppm by weight, based on the total weight of the solvent composition;
wherein the concentration of the peroxide in the solvent composition is less than 20 ppm, based on the total weight of the solvent composition; and wherein the APHA color index of the solvent composition is less than 25.

9. A glycol ether solvent composition prepared by the process of claim 8.
